(19) United States
(12) Patent Application Publication
(10) Pub. No.: US 2010/0216684 A1
(43) Pub. Date: Aug. 26, 2010

(54) PERFUME PARTICLES

(76) Inventors: Paul Ferguson, Wirral (GB); Anthony Hackett, Wirral (GB); Robert Alan Hunter, Wirral (GB); Christopher Clarkson Jones, Wirral (GB); Craig Warren Jones, Wirral (GB)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/600,713

(22) PCT Filed: May 16, 2008
(86) PCT No.: PCT/EP08/56060

§ 371 Date: Mar. 30, 2010

(30) Foreign Application Priority Data
Jun. 1, 2007 (GB) 0710369.0

(51) Int. Cl. C11D 3/50; A61K 8/11; A61Q 13/00

(52) U.S. Cl. 510/337; 512/4

(57) ABSTRACT

The invention relates to a process for the manufacture of core-shell perfume particles by emulsion polymerisation and to the products obtainable by such a process. The core of the particles comprises a perfume and the shell (which preferably comprises an aminoplast polymer) also comprises a non-ionic deposition aid (such as locust bean gum) which is substantive to textiles. The process of the invention is characterised in that, during the emulsion polymerisation step, the solids content of the polymerisation mixture does not fall below 25% wt. The particles obtained by the process are suitable for inclusion in laundry products and show significantly improved deposition and retention onto fabrics being laundered as compared with particles formed at lower solids levels.

### TECHNICAL FIELD

The present invention relates to particles comprising a perfume and a nonionic deposition aid and their uses in the formulation of laundry detergent compositions and delivery of the perfume to fabric during laundering. Laundry treatment compositions containing particles according to the invention provide deposition efficiency benefits during washing.

### BACKGROUND OF THE INVENTION

The deposition of a perfume onto a substrate, such as a fabric, is a well known method of imparting perfume properties to the substrate. In laundry applications deposition of a perfume is used, for example, during fabric treatment processes such as fabric washing and conditioning. Methods of deposition are diverse and include deposition during the wash or rinse stages of the laundry process or direct deposition before or after the wash, such as by spraying, rubbing, by use of impregnated sheets during tumble drying or water additives during steam ironing.

The perfume is often incorporated into a carrier or delivery system. Carrier systems for perfumes are typically based on encapsulation or entrapment of the perfume within a matrix.

The perfume may simply be emulsified but due to problems with poor retention or stability exist, deposition is often inefficient. Diffusion of the perfume into a carrier can require complex preparation due to required time of diffusion. Poor retention of the perfume in the matrix and subsequent poor substrate deposition are also common problems. Longevity of adherence of the perfume is inherently poor as surfactants are very efficient at combining with perfumes. Thus, a perfume which has been deposited onto a fabric can be washed-off. Also, the perfume can be leached from its carrier in the wash liquor thus becoming unavailable for deposition onto the fabric. Protection of the perfume is, therefore, required before and after it has been deposited onto a surface.

### PRIOR ART

Our co-pending patent application, PCT/EP2005/004779, unpublished at the filing date of this application, is directed towards a process, which uses miniemulsion polymerisation, for the preparation of polysaccharide/polymer conjugate particles containing a lubricant. Certain particles produced by the process and uses thereof are also disclosed. The particles facilitate deposition of the lubricant to fabric during the main wash part of a laundering process.

Our co-pending patent applications, GB 0513803.7 and GB 051805.2, both unpublished at the filing date of this application, are directed towards miniemulsion polymer particles (with and without a shell respectively) comprising a benefit agent, preferably a sugar polyester, which may be delivered to fabric during laundering. These particles give long lasting adherence of the benefit agent to fabric during laundering.

Our co-pending patent application GB 0524665.7 (WO 2007/062733) unpublished at the priority date of this application, discloses that a perfume can be efficiently deposited onto a fabric by a perfume/carrier system based on a colloidal particle comprising a polymer, perfume and a nonionic deposition aid (such as locust bean gum). The process disclosed in that application is a so-called “mini-emulsion” process in which polymer/perfume core particles were formed by evaporation of a solution of polymer and perfume in dichloromethane to produce a dispersion of perfume/polymer cores. This process is not a polymerisation reaction. Subsequently, the cores were treated with a deposition aid (LBG) in the presence of a monomer (vinyl acetate) under conditions in which the monomer would react to form an outer shell containing the deposition aid. The solids content at the end of the process was 15-16%.

Our co-pending application GB 0524659.0 (later published as WO 2007/062833) discloses encapsulates comprising a benefit agent core (preferably a perfume), one or more inner shells and an outer shell comprising a structural polymer and a polymer (for example locust bean gum) which is substantive to cellulose. At least one of said shells is impermeable to the benefit agent, and the core is formed prior to the shells. The process disclosed in that application is a two-step process and during the emulsion polymerisation step to add the outer shell the solids level is 6.5%. Throughout the specification % solids is expressed as wt % as are other percentages unless otherwise indicated.

In both WO2007/062833 and WO2007/062733 improved deposition of the perfume particles is obtained (as compared to unmodified perfume capsules) by the use of a non-ionic deposition aid. However, there is a need to further improve the deposition and to simplify the process by which the particles may be formed. As the use of perfume encapsulates adds significant cost and complexity to the process of manufacturing laundry products, the use of these encapsulates needs to significantly improve perfume delivery without adding excessive processing costs.

### DEFINITION OF THE INVENTION

The present invention is concerned with an optimised, ideally one vessel, emulsion polymerisation process for the manufacture of core/shell perfume particles which particles further comprise a non-ionic deposition aid. Unexpectedly, we have determined that when the emulsion polymerisation step is performed at a solids level of >25% wt, particles are obtained which exhibit significantly improved deposition.

Accordingly, the present invention provides a process for the manufacture of core-shell particles by emulsion polymerisation wherein the core comprises a perfume and the shell comprises a non-ionic deposition aid which is substantive to textiles, characterised in that, during the emulsion polymerisation step the solids content does not fall below 25% wt.

By performing the emulsification at higher solids content, and particularly at solids content of from 30-50% wt, preferably around 40% wt, an unexpected and significantly improved deposition of the particles obtained is obtained. In the examples given herein polymerisation at a solids level of 20% gave particles of which around 60% were deposited and retained in the wash whereas at a solids level of 40% the deposition and retention was improved, to around 75%. Thus, the loss of particles during deposition was reduced from about 40% to about 25%. Without any deposition aid present only around 30% of particles are deposited and retained during the wash.

In the context of the present invention the solids content is expressed as the wt % of organic matter present in the emulsion irrespective as to whether this is in solution or not. It excludes solvents. In the prior processes, particles were either formed at a low solids level or diluted to a low solids level during the process. The improvement obtained by the present invention is comparable to the step change obtained by the use of a deposition aid per se.

“Substantive to textiles” is a term well understood in the art and means that the non-ionic deposition aid has a specific affinity for textiles. As will be discussed in further detail below one such deposition aid is Locust Bean Gum (LBG) which is substantive to cellulose-based textiles such as cotton. Other depositions aids, such as the polyesters of dicarboxylic acids and polyols (materials of this general type are known as “soil release polymers”) have affinity for polyesters.

Production of capsules at a higher solids level also means that less liquid needs to be removed if a dry product such as a detergent powder is to be produced. It is also possible to use smaller production facilities and/or less energy and/or have a higher production throughput.

By changing the materials present during the polymerisation process it is possible to ensure that the non-ionic deposition aid is predominantly attached to the outer surface of the particles. Preferably the polymerisation is a two step process, of which both steps are performed at a solids content above 25%.

In a preferred embodiment of the invention, a first process step comprises the formation of perfume encapsulates comprising a perfume core and shell from an emulsion of perfume in an aqueous solution of a monomer in which the emulsion has a solids content of above 25%, preferably at 30-50 wt %.

Preferably, the shell is formed at least in part by step-growth polymerisation. Typically, these will be melamine/urea-formaldehyde shells formed by step-growth polymerisation of melamine/urea (or mixtures thereof) and formaldehyde monomers. In the alternative the shell can be formed by an addition polymerisation. If addition polymerisation is used then a methyl methacryl is typically used as monomer and the shells will typically comprise polymethyl-methacrylate. Alternative addition polymerisation monomers as discussed in further detail below.

It is preferred that the non-ionic deposition aid is added to the polymerisation mixture only after a shell has at least in part been formed. Typically the non-ionic deposition aid will be a polysaccharide. Suitable materials are discussed in further detail below. The preferred non-ionic deposition aid is Locust Bean Gum, which is substantive to cotton and other cellulose based materials. Other non-ionic deposition aids are known (such as those used as soil release polymers) and these provide alternatives to the Locust Bean Gum.

It is further preferred that polymerisation is concluded in the presence of a different monomer set than was present during the shell formation. Preferred monomers for the conclusion of the emulsion polymerisation are monomers with solubility in water of from 0.1 to 30 g/l. Optionally, monomers with a solubility in water of greater than 30 g/l, and/or cross linkers can also be present. Preferably, the polymerisation is concluded in the presence of at least one addition polymerisation monomer. Typically, these include the ethylenically-unsaturated monomers, particularly vinyl acetate and methyl acrylate.

Production of the particles by the two-step process can take place in a single reaction vessel. In such a process an initial emulsion polymerisation forms core-shell encapsulates of perfume with a “structural” shell. In a later stage of the emulsion polymerisation a nonionic deposition aid is added, preferably with additional monomer. This adds an outer layer to the shell, incorporating the deposition aid. Adding the deposition aid part-way through the emulsion polymerisation process allows a robust, structural inner shell to form, incorporates the deposition aid effectively and reduces digestion of the deposition aid under the conditions of emulsion polymerisation.

A second aspect of the invention provides particles obtainable by the process described above. As directly manufactured, these particles are in the form of slurry. Preferably the slurry comprises some 30-50% of organic solids.

A third aspect of the invention provides a laundry treatment composition comprising the particles of the second aspect. Use of this laundry treatment composition to provide a perfume deposition benefit to fabric is also provided.

In a fourth aspect, the invention provides the use of a particle of the second aspect to provide a perfume deposition benefit during a laundry process.

In a further aspect, the invention provides the use of a particle of the second aspect in the manufacture of a laundry treatment composition to provide a perfume deposition benefit during the laundry process.

In a still further aspect, the invention provides an aqueous wash medium comprising from 0.05 to 1 gram per litre of a particle according to the second aspect of the invention.

### DETAILED DESCRIPTION OF THE INVENTION

In order that the present invention may be further understood it is described in further detail below with reference to preferred features.

The polymer particles of the invention can comprise a wide selection of monomeric units. By “monomer units” as used herein is meant the monomeric units of the polymer chain, thus references to “a polymer particle comprising insoluble monomer units” as used herein means that the polymer particles is derived from insoluble monomers, and so forth.

As noted above, the monomer units are preferably derived from monomers which are suitable for either step growth polymerisation or addition/free radical polymerisation.

### Monomers for Step Polymerisation:

Suitable classes of such monomers are given in the group consisting of the melamine/urea/formaldehyde class, the isocyanate/diol class (preferably the polyurethanes) and polyesters. Preferred are the melamine/urea formaldehyde class and the polurethanes.

### Monomers for Addition/Free Radical Polymerisation:

Suitable classes of such monomers are given in the group consisting of olefins, ethylene, vinylaromatic monomers, esters of vinyl alcohol with mono- and di-carboxylic acids, esters of α,β-monoethylenically unsaturated mono- and dicarboxylic acids with alcohols, nitriles of α,β-monoethylenically unsaturated carboxylic acids, conjugated dienes, α,β-monoethylenically unsaturated monocarboxylic and dicarboxylic acids and their amides, methacrylic acid and its esters with alcohols and diols, acrylic acid and its esters with alcohols and diols, dimethyl or di-n-butyl maleate, and vinyl-sulfonic acid and its water-soluble salts, and mixtures thereof. The polymer particle may comprise mixtures of monomer units.

The polymer particle may optionally comprise monomers which are cross-linkers. Such crosslinkers may have at least two non-conjugated ethylenically unsaturated double bonds. Examples are alkylene glycol diacrylates and dimethacrylates. A further type of suitable cross-linking monomers are those that are conjugated, such as divinyl benzene. If present, these monomers constitute from 0.1 to 10% by weight, based on the total amount of monomers to be polymerised.

The monomers are preferably selected from: styrene; α-methylstyrene; o-chlorostyrene; vinyl acetate; vinyl propionate; vinyl n-butyrate; esters of acrylic, methacrylic, maleic, fumaric or itaconic acid with methyl, ethyl, n-butyl, isobutyl, n-hexyl and 2-ethylhexyl alcohol; 1,3-butadiene; 2,3 dimethyl butadiene; and isoprene. The preferred monomers are vinyl acetate and methyl acrylate.

Optionally, the monomers are used as co-polymers with one or more of acrylic acid, methacrylic acid, maleic acid, fumaric acid, itaconic acid, poly(alkylene oxide)monoacrylates and monomethacrylates, N-vinyl-pyrrolidone, methacrylic and acrylic acid, 2-hydroxyethyl acrylates and methacrylates, glycerol acrylates and methacrylates, poly(ethylene glycol) methacrylates and acrylates, n-vinyl pyrrolidone, acryloyl morpholine, vinyl formamide, n-vinyl acetamide and vinyl caprolactone, acrylonitrile (71 g/l), acrylamide, and methacrylamide at levels of less than 10% by weight of the monomer unit content of the particle; 2-(dimethylamino) ethyl methacrylate, 2-(diethylamino) ethyl methacrylate, 2-(tert-butylamino) ethyl methacrylate, 2-aminoethyl methacrylate, 2-(2-oxo-1-imidazolidinyl)ethyl methacrylate, vinyl pyridine, vinyl carbazole, vinyl imidazole, vinyl aniline, and their cationic forms after treatment with alkyl halides;

Optional cross linkers include vinyltoluenes, divinyl benzene, ethylene glycol diacrylate, 1,2-propylene glycol diacrylate, 1,3-propylene glycol diacrylate, 1,3-butylene glycol diacrylate, 1,4-butylene glycol diacrylates, ethylene glycol dimethacrylate, 1,2-propylene glycol dimethacrylate, 1,3-propylene glycol dimethacrylate, 1,3-butylene glycol dimethacrylate, 1,4-butylene glycol dimethacrylate, divinylbenzene, vinyl methacrylate, vinyl acrylate, allyl methacrylate, allyl acrylate, diallyl maleate, diallyl fumarate, methylenebisacrylamide, cyclopentadienyl acrylate, and triallyl cyanurate.

It is preferable that the ratio of the monomers used in the shell formation and those used in deposition aid attachment are the ratio of 20:1 to 1:1 (as shellformer:deposition linker). Preferably, the ratio is 5:1-2:1, more preferably 4:1-2:1 as better particle deposition on fabric is found as the ratio approaches 2:1.

### Non-Ionic Deposition Aid

Preferably, the deposition aid is a polysaccharide. The polysaccharide preferably has a 8-1,4-linked backbone.

Preferably the polysaccharide is a cellulose, a cellulose derivative, or another β-1,4-linked polysaccharide having an affinity for cellulose, such as polymannan, polyglucan, polyglucomannan, polyxyloglucan and polygalactomannan or a mixture thereof. More preferably, the polysaccharide is selected from the group consisting of polyxyloglucan and polygalactomannan. For example, preferred polysaccharides are locust bean gum, tamarind xyloglucan, guar gum or mixtures thereof. Most preferably, the deposition aid is locust bean gum.

Polysaccharides can act as thickeners when added to an emulsion system, such as that employed herein. One problem with increased viscosity is it can determine how much polysaccharide one can coat a capsule with, and on a large scale flexibility of processing can be compromised. We have determined that selection of the polysaccharide type can modify the viscosity.

Preferably, the polysaccharide backbone has only β-1,4 linkages. Optionally, the polysaccharide has linkages in addition to the β-1,4 linkages, such as β-1,3 linkages. Thus, optionally some other linkages are present. Polysaccharide backbones which include some material which is not a saccharide ring are also within the ambit of the present invention (whether terminal or within the polysaccharide chain).

The polysaccharide may be straight or branched. Many naturally occurring polysaccharides have at least some degree of branching, or at any rate at least some saccharide rings are in the form of pendant side groups (which are therefore not in themselves counted in determining the degree of substitution) on a main polysaccharide backbone.

Preferably, the polysaccharide is present at levels of between 0.1% to 10% w/w by weight of the total amount of the particle.

The deposition aid, which is preferably a polysaccharide, is attached to the by means of a covalent bond, entanglement or strong adsorption, preferably by a covalent bond or entanglement and most preferably by means of a covalent bond. By entanglement as used herein is meant that the deposition aid is adsorbed onto the particle as the polymerisation proceeds and the particle grows in size, part of the adsorbed deposition aid becomes buried within the interior of the particle. Hence at the end of the polymerisation, part of the deposition aid is entrapped and bound in the polymer matrix of the particle, whilst the remainder is free to extend into the aqueous phase.

By strong adsorption as used herein is meant strong adsorption of the deposition aid to the surface of the particle; such adsorption can, for example, occur due to hydrogen bonding, Van Der Waals or electrostatic attraction between the deposition aid and the particle.

The deposition aid is thus mainly attached to the particle surface and is not, to any significant extent, distributed throughout the internal bulk of the particle. This is distinct from graft copolymers in which e.g. a polysaccharide may be grafted along the length of a polymer chain. A particle which is formed from a graft copolymer would, therefore, contain polysaccharide throughout the internal bulk of the particle as well as on the particle surface and the present invention is not intended to cover such a particle. Thus the particle which is produced when using a polysaccharide as the deposition aid according to the process of the invention can be thought of as a “hairy particle”, which is different from a graft copolymer. This feature of the invention provides significant cost reduction opportunities for the manufacturer as much less deposition aid is required to achieve the same level of activity as systems which utilise polysaccharide copolymers.

Other types of particle surface morphology may be produced when a deposition aid is attached to the particle of the invention. For example, where a polysaccharide attaches to the particle surface in multiple places, loops may result, or the deposition aid may be in the form of a swollen polymer layer at the particle surface.

In one particularly preferred aspect of the invention the deposition aid is grafted with a polymer prior to addition to the reaction mixture containing the particles.

### Perfumes

The perfume is typically present in an amount of from 10-85% by total weight of the particle, preferably from 20 to 75% by total weight of the particle.

The perfume suitably has a molecular weight of from 50 to 500.

Useful components of the perfume include materials of both natural and synthetic origin. They include single compounds and mixtures. Specific examples of such components may be found in the current literature, e.g., in Fenaroli's Handbook of Flavor Ingredients, 1975, CRC Press; Synthetic Food Adjuncts, 1947 by M. B. Jacobs, edited by Van Nostrand; or Perfume and Flavor Chemicals by S. Arctander 1969, Montclair, N.J. (USA). These substances are well known to the person skilled in the art of perfuming, flavoring, and/or aromatizing consumer products, i.e., of imparting an odor and/or a flavor or taste to a consumer product traditionally perfumed or flavored, or of modifying the odor and/or taste of said consumer product.

By perfume in this context is not only meant a fully formulated product fragrance, but also selected components of that fragrance, particularly those which are prone to loss, such as the so-called ‘top notes’.

Top notes are defined by Poucher (Journal of the Society of Cosmetic Chemists 6(2):80 [1955]). Examples of well known top-notes include citrus oils, linalool, linalyl acetate, lavender, dihydromyrcenol, rose oxide and cis-3-hexanol. Top notes typically comprise 15-25% wt of a perfume composition and in those embodiments of the invention which contain an increased level of top-notes it is envisaged at that least 20% wt would be present within the encapsulate.

Typical perfume components which it is advantageous to encapsulate, include those with a relatively low boiling point, preferably those with a boiling point of less than 300, preferably 100-250 Celsius.

It is also advantageous to encapsulate perfume components which have a low Log P (ie. those which will be partitioned into water), preferably with a Log P of less than 3.0. These materials, of relatively low boiling point and relatively low Log P have been called the “delayed blooming” perfume ingredients and include the following materials:

Allyl Caproate, Amyl Acetate, Amyl Propionate, Anisic Aldehyde, Anisole, Benzaldehyde, Benzyl Acetate, Benzyl Acetone, Benzyl Alcohol, Benzyl Formate, Benzyl Iso Valerate, Benzyl Propionate, Beta Gamma Hexenol, Camphor Gum, Laevo-Carvone, d-Carvone, Cinnamic Alcohol, Cinamyl Formate, Cis-Jasmone, cis-3-Hexenyl Acetate, Cuminic Alcohol, Cyclal C, Dimethyl Benzyl Carbinol, Dimethyl Benzyl Carbinol Acetate, Ethyl Acetate, Ethyl Aceto Acetate, Ethyl Amyl Ketone, Ethyl Benzoate, Ethyl Butyrate, Ethyl Hexyl Ketone, Ethyl Phenyl Acetate, Eucalyptol, Eugenol, Fenchyl Acetate, Flor Acetate (tricyclo Decenyl Acetate), Frutene (tricyclco Decenyl Propionate), Geraniol, Hexenol, Hexenyl Acetate, Hexyl Acetate, Hexyl Formate, Hydratropic Alcohol, Hydroxycitronellal, Indone, Isoamyl Alcohol, Iso Menthone, Isopulegyl Acetate, Isoquinolone, Ligustral, Linalool, Linalool Oxide, Linalyl Formate, Menthone, Menthyl Acetphenone, Methyl Amyl Ketone, Methyl Anthranilate, Methyl Benzoate, Methyl Benzyl Acetate, Methyl Eugenol, Methyl Heptenone, Methyl Heptine Carbonate, Methyl Heptyl Ketone, Methyl Hexyl Ketone, Methyl Phenyl Carbinyl Acetate, Methyl Salicylate, Methyl-N-Methyl Anthranilate, Nerol, Octalactone, Octyl Alcohol, p-Cresol, p-Cresol Methyl Ether, p-Methoxy Acetophenone, p-Methyl Acetophenone, Phenoxy Ethanol, Phenyl Acetaldehyde, Phenyl Ethyl Acetate, Phenyl Ethyl Alcohol, Phenyl Ethyl Dimethyl Carbinol, Prenyl Acetate, Propyl Bornate, Pulegone, Rose Oxide, Safrole, 4-Terpinenol, Alpha-Terpinenol, and/or Viridine

It is commonplace for a plurality of perfume components to be present in a formulation. In the encapsulates of the present invention it is envisaged that there will be four or more, preferably five or more, more preferably six or more or even seven or more different perfume components from the list given of delayed blooming perfumes given above present in the encapsulated perfume.

Another group of perfumes with which the present invention can be applied are the so-called ‘aromatherapy’ materials. These include many components also used in perfumery, including components of essential oils such as Clary Sage, Eucalyptus, Geranium, Lavender, Mace Extract, Neroli, Nutmeg, Spearmint, Sweet Violet Leaf and Valerian. By means of the present invention these materials can be transferred to textile articles that will be worn or otherwise come into contact with the human body (such as handkerchiefs and bed-linen).

### Process Details

As noted above the process for the preparation of the particles is preferably a two step process in which the first step forms a capsule and the second step applies a coating to it. The first step can either be step-growth or addition polymerisation and the second step is preferably addition polymerisation.

It is particularly preferably that the first step uses monomers selected from melamine/urea-formaldehyde or methyl-methacrylate or isocyanate/diol, and the second step uses monomers selected from vinyl acetate and/or methyl acrylate. It is particular preferred that the non-ionic deposition aid is not added until the second step.

For step-growth polymerisation some heating is generally necessary to cause polymerisation to proceed. Initiators and chain transfer agents may also be present in the polymerisation mixture where use is made of any addition polymerisation. Those skilled in the art will recognise that a chemical initiator will generally be required for addition polymerisation but that there are instances in which alternative forms of initiation will be possible, e.g. ultrasonic initiation or initiation by irradiation.

The initiator is preferably a chemical or chemicals capable of forming free radicals. Typically, free radicals can be formed either by homolytic scission (i.e. homolysis) of a single bond or by single electron transfer to or from an ion or molecule (e.g. redox reactions). Suitably, in context of the invention, homolysis may be achieved by the application of heat (typically in the range of from 50 to 100° C.). Some examples of suitable initiators in this class are those possessing peroxide (—O—O—) or azo (—N═N—) groups, such as benzoyl peroxide, t-butyl peroxide, hydrogen peroxide, azobisisobutyronitrile and ammonium persulphate. Homolysis may also be achieved by the action of radiation (usually ultraviolet), in which case it is termed photolysis. Examples are the dissociation of 2,2′-azobis(2-cyanopropane) and the formation of free radicals from benzophenone and benzoin. Redox reactions can also be used to generate free radicals. In this case an oxidising agent is paired with a reducing agent which then undergo a redox reaction. Some examples of appropriate pairs in the context of the invention are ammonium persulphate/sodium metabisulphite, cumyl hydroperoxide/ferrous ion and hydrogen peroxide/ascorbic acid.

Preferred initiators are selected from the following:
Homolytic: benzoyl peroxide, t-butyl peroxide, hydrogen peroxide, azobisisobutyronitrile, ammonium persulphate, 2,2′-azobis(cyanopropane), benzophenone, benzoin,
Redox: ammonium persulphate/sodium metabisulphite mixture, cumyl hydroperoxide/ferrous ion mixture and/or hydrogen peroxide/ascorbic acid mixture.

Preferred initiators are ammonium persulphate and hydrogen peroxide/ascorbic acid mixture. The preferred level of initiator is in the range of from 0.1 to 5.0% w/w by weight of monomer, more preferably, the level is in the range of from 1.0 to 3.0% w/w by weight of monomer.

Chain transfer agents can optionally be used. A chain transfer agent contains very labile hydrogen atoms that are easily abstracted by a propagating polymer chain. This terminates the polymerisation of the growing polymer, but generates a new reactive site on the chain transfer agent that can then proceed to initiate further polymerisation of the remaining monomer. Chain transfer agents in the context of the invention typically contain thiol (mercaptan) functionality and can be represented by the general chemical formula RS—H, such as n-dodecyl mercaptan and 2-mercaptoethanol. Preferred chain transfer agents are monothioglycerol and n-dodecyl mercaptan, used at levels of, preferably from 0 to 5% w/w based on the weight of the monomer and more preferably at a level of 0.25% w/w based on the weight of the monomer.

The preferred product of such a process is a slurry or dispersion comprising some 30-50% of solids.

### Laundry Treatment Compositions

The polymer particles of the invention may be incorporated into laundry compositions. This may be done by mixing the slurry/dispersion products as mentioned above with some or all of the other components of the composition, preferably by spraying onto the components. Advantageously, the slurry/dispersion need not be dried extensively (if at all) and this reduces perfume losses.

The polymer particles are typically included in said compositions at levels of from 0.001% to 10%, preferably from 0.005% to 5%, most preferably from 0.01% to 3% by weight of the total composition.

The active ingredient in the compositions is preferably a surface active agent or a fabric conditioning agent. More than one active ingredient may be included. For some applications a mixture of active ingredients may be used.

The compositions of the invention may be in any physical form e.g. a solid such as a powder or granules, a tablet, a solid bar, a paste, gel or liquid, especially, an aqueous based liquid. In particular the compositions may be used in laundry compositions, especially in liquid, powder or tablet laundry composition.

The compositions of the present invention are preferably laundry compositions, especially main wash (fabric washing) compositions or rinse-added softening compositions. The main wash compositions may include a fabric softening agent and the rinse-added fabric softening compositions may include surface-active compounds, particularly non-ionic surface-active compounds.

The detergent compositions of the invention may contain a surface-active compound (surfactant) which may be chosen from soap and non-soap anionic, cationic, non-ionic, amphoteric and zwitterionic surface-active compounds and mixtures thereof. Many suitable surface-active compounds are available and are fully described in the literature, for example, in “Surface-Active Agents and Detergents”, Volumes I and II, by Schwartz, Perry and Berch.

The preferred detergent-active compounds that can be used are soaps and synthetic non-soap anionic, and non-ionic compounds.

The compositions of the invention may contain linear alkylbenzene sulphonate, particularly linear alkylbenzene sulphonates having an alkyl chain length of from C8 to C15. It is preferred if the level of linear alkylbenzene sulphonate is from 0 wt % to 30 wt %, more preferably from 1 wt % to 25 wt %, most preferably from 2 wt % to 15 wt %, by weight of the total composition.

The compositions of the invention may contain other anionic surfactants in amounts additional to the percentages quoted above. Suitable anionic surfactants are well-known to those skilled in the art. Examples include primary and secondary alkyl sulphates, particularly C8 to C15 primary alkyl sulphates; alkyl ether sulphates; olefin sulphonates; alkyl xylene sulphonates; dialkyl sulphosuccinates; and fatty acid ester sulphonates. Sodium salts are generally preferred.

The compositions of the invention may also contain non-ionic surfactant. Nonionic surfactants that may be used include the primary and secondary alcohol ethoxylates, especially the C8 to C20 aliphatic alcohols ethoxylated with an average of from 1 to 20 moles of ethylene oxide per mole of alcohol, and more especially the C10 to C15 primary and secondary aliphatic alcohols ethoxylated with an average of from 1 to 10 moles of ethylene oxide per mole of alcohol. Non-ethoxylated nonionic surfactants include alkylpolyglycosides, glycerol monoethers, and polyhydroxyamides (glucamide).

It is preferred if the level of non-ionic surfactant is from 0 wt % to 30 wt %, preferably from 1 wt % to 25 wt %, most preferably from 2 wt % to 15 wt %, by weight of the total composition.

Any conventional fabric conditioning agent may be used in the compositions of the present invention. The conditioning agents may be cationic or non-ionic. If the fabric conditioning compound is to be employed in a main wash detergent composition the compound will typically be non-ionic. For use in the rinse phase, typically they will be cationic. They may for example be used in amounts from 0.5% to 35%, preferably from 1% to 30% more preferably from 3% to 25% by weight of the composition.

Suitable cationic fabric softening compounds are substantially water-insoluble quaternary ammonium materials comprising a single alkyl or alkenyl long chain having an average chain length greater than or equal to C20 or, more preferably, compounds comprising a polar head group and two alkyl or alkenyl chains having an average chain length greater than or equal to C14. Preferably the fabric softening compounds have two long chain alkyl or alkenyl chains each having an average chain length greater than or equal to C16. Most preferably at least 50% of the long chain alkyl or alkenyl groups have a chain length of C18 or above. It is preferred if the long chain alkyl or alkenyl groups of the fabric softening compound are predominantly linear.

Quaternary ammonium compounds having two long-chain aliphatic groups, for example, distearyldimethyl ammonium chloride and di(hardened tallow alkyl)dimethyl ammonium chloride, are widely used in commercially available rinse conditioner compositions. Other examples of these cationic compounds are to be found in “Surfactants Science Series” volume 34 ed. Richmond 1990, volume 37 ed. Rubingh 1991 and volume 53 eds. Cross and Singer 1994, Marcel Dekker Inc. New York”.

Any of the conventional types of such compounds may be used in the compositions of the present invention.

The fabric softening compounds are preferably compounds that provide excellent softening, and are characterised by a chain melting Lβ to Lα transition temperature greater than 250° C., preferably greater than 350° C., most preferably greater than 450° C. This Lβ to Lα transition can be measured by differential scanning calorimetry as defined in “Handbook of Lipid Bilayers”, D Marsh, CRC Press, Boca Raton, Fla., 1990 (pages 137 and 337).

Substantially water-insoluble fabric softening compounds are defined as fabric softening compounds having a solubility of less than 1×10⁻³ wt % in demineralised water at 20° C.

Preferably the fabric softening compounds have a solubility of less than 1×10⁻⁴ wt %, more preferably from less than 1×10⁻⁸ to 1×10⁻⁶ wt %.

Especially preferred are cationic fabric softening compounds that are water-insoluble quaternary ammonium materials having two C12-22 alkyl or alkenyl groups connected to the molecule via at least one ester link, preferably two ester links. Di(tallowoxyloxyethyl)dimethyl ammonium chloride and/or its hardened tallow analogue is an especially preferred compound of this class.

A second preferred type comprises those derived from triethanolamine (hereinafter referred to as ‘TEA quats’) as described in for example U.S. Pat. No. 3,915,867. Suitable materials are, for example, N-methyl-N,N,N-triethanolamine ditallowester or di-hardened-tallowester quaternary ammonium chloride or methosulphate. Examples of commercially available TEA quats include Rewoquat WE18 and Rewoquat WE20, both partially unsaturated (ex. WITCO), Tetranyl AOT-1, fully saturated (ex. KAO) and Stepantex VP 85, fully saturated (ex. Stepan).

It is advantageous if the quaternary ammonium material is biologically biodegradable.

It is also possible to include certain mono-alkyl cationic surfactants which can be used in main-wash compositions for fabrics. Cationic surfactants that may be used include quaternary ammonium salts of the general formula R1R2R3R4N+X— wherein the R groups are long or short hydrocarbon chains, typically alkyl, hydroxyalkyl or ethoxylated alkyl groups, and X is a counter-ion (for example, compounds in which R1 is a C8-C22 alkyl group, preferably a C8-C10 or C12-C14 alkyl group, R2 is a methyl group, and R3 and R4, which may be the same or different, are methyl or hydroxyethyl groups); and cationic esters (for example, choline esters).

The choice of surface-active compound (surfactant), and the amount present, will depend on the intended use of the detergent composition. In fabric washing compositions, different surfactant systems may be chosen, as is well known to the skilled formulator, for handwashing products and for products intended for use in different types of washing machine.

The total amount of surfactant present will also depend on the intended end use and may be as high as 60 wt %, for example, in a composition for washing fabrics by hand. In compositions for machine washing of fabrics, an amount of from 5 to 40 wt % is generally appropriate. Typically the compositions will comprise at least 2 wt % surfactant e.g. 2-60%, preferably 15-40% most preferably 25-35%, by weight of the composition.

Detergent compositions suitable for use in most automatic fabric washing machines generally contain anionic non-soap surfactant, or non-ionic surfactant, or combinations of the two in any suitable ratio, optionally together with soap.

The compositions of the invention, when used as main wash fabric washing compositions, will generally also contain one or more detergency builders. The total amount of detergency builder in the compositions will typically range from 5 to 80 wt %, preferably from 10 to 60 wt %, by weight of the compositions.

Inorganic builders that may be present include sodium carbonate, if desired in combination with a crystallisation seed for calcium carbonate, as disclosed in GB 1 437 950 (Unilever); crystalline and amorphous aluminosilicates, for example, zeolites as disclosed in GB 1 473 201 (Henkel), amorphous aluminosilicates as disclosed in GB 1 473 202 (Henkel) and mixed crystalline/amorphous aluminosilicates as disclosed in GB 1 470 250 (Procter & Gamble); and layered silicates as disclosed in EP 164 514B (Hoechst). Inorganic phosphate builders, for example, sodium orthophosphate, pyrophosphate and tripolyphosphate are also suitable for use with this invention.

The compositions of the invention preferably contain an alkali metal, preferably sodium, aluminosilicate builder. Sodium aluminosilicates may generally be incorporated in amounts of from 10 to 70% by weight (anhydrous basis), preferably from 25 to 50 wt %.

The alkali metal aluminosilicate may be either crystalline or amorphous or mixtures thereof, having the general formula: 0.8-1.5 Na₂O.Al₂O₃. 0.8-6 SiO₂

These materials contain some bound water and are required to have a calcium ion exchange capacity of at least 50 mg CaO/g. The preferred sodium aluminosilicates contain 1.5-3.5 SiO2 units (in the formula above). Both the amorphous and the crystalline materials can be prepared readily by reaction between sodium silicate and sodium aluminate, as amply described in the literature. Suitable crystalline sodium aluminosilicate ion-exchange detergency builders are described, for example, in GB 1 429 143 (Procter & Gamble). The preferred sodium aluminosilicates of this type are the well-known commercially available zeolites A and X, and mixtures thereof.

The zeolite may be the commercially available zeolite 4A now widely used in laundry detergent powders. However, according to a preferred embodiment of the invention, the zeolite builder incorporated in the compositions of the invention is maximum aluminium zeolite P (zeolite MAP) as described and claimed in EP 384 070A (Unilever). Zeolite MAP is defined as an alkali metal aluminosilicate of the zeolite P type having a silicon to aluminium weight ratio not exceeding 1.33, preferably within the range of from 0.90 to 1.33, and more preferably within the range of from 0.90 to 1.20.

Especially preferred is zeolite MAP having a silicon to aluminium weight ratio not exceeding 1.07, more preferably about 1.00. The calcium binding capacity of zeolite MAP is generally at least 150 mg CaO per g of anhydrous material.

Organic builders that may be present include polycarboxylate polymers such as polyacrylates, acrylic/maleic copolymers, and acrylic phosphinates; monomeric polycarboxylates such as citrates, gluconates, oxydisuccinates, glycerol mono-, di and trisuccinates, carboxymethyloxy succinates, carboxy-methyloxymalonates, dipicolinates, hydroxyethyliminodi-acetates, alkyl- and alkenylmalonates and succinates; and sulphonated fatty acid salts. This list is not intended to be exhaustive.

Especially preferred organic builders are citrates, suitably used in amounts of from 5 to 30 wt %, preferably from 10 to 25 wt %; and acrylic polymers, more especially acrylic/maleic copolymers, suitably used in amounts of from 0.5 to 15 wt %, preferably from 1 to 10 wt %.

Builders, both inorganic and organic, are preferably present in alkali metal salt, especially sodium salt, form.

Compositions according to the invention may also suitably contain a bleach system. Fabric washing compositions may desirably contain peroxy bleach compounds, for example, inorganic persalts or organic peroxyacids, capable of yielding hydrogen peroxide in aqueous solution.

Suitable peroxy bleach compounds include organic peroxides such as urea peroxide, and inorganic persalts such as the alkali metal perborates, percarbonates, perphosphates, persilicates and persulphates. Preferred inorganic persalts are sodium perborate monohydrate and tetrahydrate, and sodium percarbonate.

Especially preferred is sodium percarbonate having a protective coating against destabilisation by moisture. Sodium percarbonate having a protective coating comprising sodium metaborate and sodium silicate is disclosed in GB 2 123 044B (Kao).

The peroxy bleach compound is suitably present in an amount of from 0.1 to 35 wt %, preferably from 0.5 to 25 wt %. The peroxy bleach compound may be used in conjunction with a bleach activator (bleach precursor) to improve bleaching action at low wash temperatures. The bleach precursor is suitably present in an amount of from 0.1 to 8 wt %, preferably from 0.5 to 5 wt %.

Preferred bleach precursors are peroxycarboxylic acid precursors, more especially peracetic acid precursors and pernoanoic acid precursors. Especially preferred bleach precursors suitable for use in the present invention are N,N,N′,N′,-tetracetyl ethylenediamine (TAED) and sodium nonanoyloxybenzene sulphonate (SNOBS). The novel quaternary ammonium and phosphonium bleach precursors disclosed in U.S. Pat. No. 4,751,015 and U.S. Pat. No. 4,818,426 (Lever Brothers Company) and EP 402 971A (Unilever), and the cationic bleach precursors disclosed in EP 284 292A and EP 303 520A (Kao) are also of interest.

The bleach system can be either supplemented with or replaced by a peroxyacid. Examples of such peracids can be found in U.S. Pat. No. 4,686,063 and U.S. Pat. No. 5,397,501 (Unilever). A preferred example is the imido peroxycarboxylic class of peracids described in EP A 325 288, EP A 349 940, DE 382 3172 and EP 325 289. A particularly preferred example is phthalimido peroxy caproic acid (PAP). Such peracids are suitably present at 0.1-12%, preferably 0.5-10%.

A bleach stabiliser (transition metal sequestrant) may also be present. Suitable bleach stabilisers include ethylenediamine tetra-acetate (EDTA), the polyphosphonates such as Dequest (Trade Mark) and non-phosphate stabilisers such as EDDS (ethylene diamine di-succinic acid). These bleach stabilisers are also useful for stain removal especially in products containing low levels of bleaching species or no bleaching species.

An especially preferred bleach system comprises a peroxy bleach compound (preferably sodium percarbonate optionally together with a bleach activator), and a transition metal bleach catalyst as described and claimed in EP 458 397A, EP 458 398A and EP 509 787A (Unilever).

The compositions according to the invention may also contain one or more enzyme(s).

Suitable enzymes include the proteases, amylases, cellulases, oxidases, peroxidases and lipases usable for incorporation in detergent compositions. Preferred proteolytic enzymes (proteases) are, catalytically active protein materials which degrade or alter protein types of stains when present as in fabric stains in a hydrolysis reaction. They may be of any suitable origin, such as vegetable, animal, bacterial or yeast origin.

Proteolytic enzymes or proteases of various qualities and origins and having activity in various pH ranges of from 4-12 are available and can be used in the instant invention. Examples of suitable proteolytic enzymes are the subtilisins which are obtained from particular strains of B. Subtilis B. licheniformis, such as the commercially available subtilisins Maxatase (Trade Mark), as supplied by Genencor International N.V., Delft, Holland, and Alcalase (Trade Mark), as supplied by Novozymes Industri A/S, Copenhagen, Denmark.

Particularly suitable is a protease obtained from a strain of Bacillus having maximum activity throughout the pH range of 8-12, being commercially available, e.g. from Novozymes Industri A/S under the registered trade-names Esperase (Trade Mark) and Savinase (Trade-Mark). The preparation of these and analogous enzymes is described in GB 1 243 785. Other commercial proteases are Kazusase (Trade Mark obtainable from Showa-Denko of Japan), Optimase (Trade Mark from Miles Kali-Chemie, Hannover, West Germany), and Superase (Trade Mark obtainable from Pfizer of U.S.A.).

Detergency enzymes are commonly employed in granular form in amounts of from about 0.1 to about 3.0 wt %. However, any suitable physical form of enzyme may be used.

The compositions of the invention may contain alkali metal, preferably sodium carbonate, in order to increase detergency and ease processing. Sodium carbonate may suitably be present in amounts ranging from 1 to 60 wt %, preferably from 2 to 40 wt %. However, compositions containing little or no sodium carbonate are also within the scope of the invention.

Powder flow may be improved by the incorporation of a small amount of a powder structurant, for example, a fatty acid (or fatty acid soap), a sugar, an acrylate or acrylate/maleate copolymer, or sodium silicate. One preferred powder structurant is fatty acid soap, suitably present in an amount of from 1 to 5 wt %.

Other materials that may be present in detergent compositions of the invention include sodium silicate; antiredeposition agents such as cellulosic polymers; soil release polymers; inorganic salts such as sodium sulphate; or lather boosters as appropriate; proteolytic and lipolytic enzymes; dyes; coloured speckles; fluorescers and decoupling polymers. This list is not intended to be exhaustive.

The detergent composition when diluted in the wash liquor (during a typical wash cycle) will typically give a pH of the wash liquor from 7 to 10.5 for a main wash detergent.

Particulate detergent compositions are suitably prepared by spray-drying a slurry of compatible heat-insensitive ingredients, and then spraying on or post-dosing those ingredients unsuitable for processing via the slurry. The skilled detergent formulator will have no difficulty in deciding which ingredients should be included in the slurry and which should not.

Particulate detergent compositions of the invention preferably have a bulk density of at least 400 g/litre, more preferably at least 500 g/litre. Especially preferred compositions have bulk densities of at least 650 g/litre, more preferably at least 700 g/litre.

Such powders may be prepared either by post-tower densification of spray-dried powder, or by wholly non-tower methods such as dry mixing and granulation; in both cases a high-speed mixer/granulator may advantageously be used. Processes using high-speed mixer/granulators are disclosed, for example, in EP 340 013A, EP 367 339A, EP 390 251A and EP 420 317A (Unilever).

Liquid detergent compositions can be prepared by admixing the essential and optional ingredients thereof in any desired order to provide compositions containing components in the requisite concentrations. Liquid compositions according to the present invention can also be in compact form which means it will contain a lower level of water compared to a conventional liquid detergent.

### Product Forms

The composition of the invention may be in the form of a liquid, solid (e.g. powder or tablet), a gel or paste, spray, stick, bar or a foam or mousse. Examples include a soaking product, a rinse treatment (e.g. conditioner or finisher) or a main-wash product. Compositions suitable for direct application are preferred, such as gel or paste, spray, stick, bar, foam or mousse. The means for manufacturing any of the product forms are well known in the art. If the polymer particles are to be incorporated in a powder (optionally the powder to be tableted), and whether or not pre-emulsified, they are optionally included in a separate granular component.

### Treatment

The treatment of a substrate with the material of the invention can be made by any suitable method such as washing, soaking or rinsing of the substrate but preferably by direct application such as spraying, rubbing, spotting, smearing, etc.

The treatment may involve contacting the substrate with an aqueous medium comprising the material of the invention.

The treatment may be provided as a spray composition e.g., for domestic (or industrial) application to fabric in a treatment separate from a conventional domestic laundering process. Suitable spray dispensing devices are disclosed in WO 96/15310 (Procter & Gamble). Alternatively, the composition may be applied through the irons water tank, a separate reservoir or a spray cartridge in an iron, as described in EP 1201816 and WO 99/27176.

In order that the present invention may be further understood and carried forth into practice it will be further described with reference to the following examples:

### EXAMPLES

Pre-formed melamine/formaldehyde perfume encapsulates were obtained from BPMW Ltd as a dispersion with a solids content of 53.2% and a size of 10-20 μm.

Perfume capsules of 5 and 30 micron were also used (prepared via emulsion polymerisation according to known literature methods). The 30 micron perfume capsules consisted of 75-76% perfume and 24-23% shell (M-F) and were used in the form of a dispersion at around 50% solids content. All types of capsule were, unless stated otherwise, modified by the addition of LBG.

In washing experiments particle deposition was measured by turbidity as follows:

### a) Preparation of Stock Solutions:

Surfactant Stock: (10 g/L 50:50 LAS:A7) was prepared by dissolving Linear Alkyl Benzene Sulphonate (9.09 g LAS (55% Active)) and Synperonic A7 (5 g) in de-ionised water to a total of 1 litre.

Base Buffer Stock: (0.1 M) was prepared by dissolving Sodium Carbonate (7.5465 g) and Sodium Hydrogen Carbonate (2.4195 g) in de-ionised water to a total of 1 litre.

### b) Reparation of the Wash Liquor:

Base Buffer Stock (12.5 ml) and surfactant stock (12.5 ml) were added to a 500 ml Linitest pot and 100 ml de-ionised water was added to produce a wash liquor buffered at pH 10.5 and containing 1 g/L surfactant (50:50 LAS:A7).

### c) Simulated Wash:

0.05 g (400 ppm on wash liquor) of polymer particles: Unmodified capsules (5-30 μm) were each added to the linitest pots containing wash liquor and agitated slightly to ensure mixing. (Washes were done in duplicate for each sample and results averaged). A 5 ml aliquot was taken from each and the Absorbance at 400 nm recorded using a 1 cm cuvette. This absorbance value represents 100% particles in the wash solution prior to the simulated simulated wash process.

### d) Linitest Equipment and Procedure:

A section of unfluoresced cotton measuring 20 cm by 20 cm was placed into each linitest pot containing the wash liquor and polymer particles and the pot was sealed.

The Linitest is a laboratory scale washing machine (Ex. Heraeus). The equipment is designed and built to comply with the requirements for international standard test specifications. It is used for small scale detergency and stain removal testing particularly when low liquor to cloth ratios are required.

There are various models of the Linitest commercially available. The model used in this case has a single rotation speed of 40 rpm. The carrier is capable of accommodating twelve 500 ml steel containers and can be operated at temperatures up to 100° C.

The Linitest comprises a 20 litre tank, control system and drive mechanism. Permanent thermostatically controlled tubular heating elements in the base of the tank heat the bath liquor to the required temperature. The stainless steel construction throughout ensures efficient heat transfer to the specimen containers that are mounted on a rotating horizontal carrier driven by a geared motor. The rotating movement of the carrier ‘throws’ the liquid from one end of the container to the other in a continuous action. This movement simulates the mechanical washing process and additional mechanical action can be obtained by using steel ball bearings or discs.

The Linitest pots were attached to the Linitester cradle and rotated 45 minutes at 40° C. to simulate the main wash.

The cloths were then removed and wrung by hand and a 5 ml aliquot of the remaining wash liquor was taken and the absorbance at 400 nm measured using a 1 cm cuvette as before. From interpolation of the initial calibration curve, the concentration of the particles remaining the liquor after the wash could be determined and hence the level deposited (wash deposition) on the cloth could be determined by difference.

The Linitest pots were then thoroughly rinsed and the ‘wrung’ cloths returned to the pots and 125 ml of de-ionised water was added. The Linitester bath water was drained and the pots attached to the cradle and rotated for 10 minutes at ambient temperature (˜20° C.) to simulate a rinse procedure. The clothes were then removed and wrung by hand. A 5 ml aliquot of the rinse solution was taken and the absorbance at 400 nm determined. As before interpolation of the initial calibration plot allowed the particle concentration removed from the cloth during the rinse to be determined and by comparison to the initial level deposited prior to the rinse, the percentage loss from the cloth could be determined. This procedure was repeated a further two times to simulate and determine losses from the second and third rinse.

In several of the examples given below the examples have been simplified by using pre-formed capsules which had been manufactured under conditions in which the solids level was above 25%. Several of the examples concentrate on the second stage of the emulsion polymerisation in which the non-ionic deposition aid is added to the capsules.

### Example 1

### Addition of LBG to Capsules Prepared at ˜50% Solids

Locust bean gum (11.2 g) was dissolved in hot (70-80° C.) de-ionised water (739.14 g) by mixing with a high speed homogeniser (Silverson) at 10,000 rpm for 10 minutes until completely solubilised. The solution was then allowed to cool to room temperature under static conditions. It was then transferred to a reaction vessel fitted with an overhead stirrer, condenser, thermocouple (attached to heating mantle) and nitrogen inlet.

Perfume encapsulates (1894.7 g, 53.2% solids as obtained from BPMW) and vinyl acetate (112 g) were added, and the contents purged with nitrogen for 10 minutes, after which point the vessel and contents were left over a nitrogen blanket for the duration of the reaction. The temperature was then raised to 70° C., and aqueous ascorbic acid solution (2.8 g in 25 g de-ionised water) together with aqueous hydrogen peroxide solution (8 g, 35% active) were added to initiate the polymerisation.

After 90 minutes a further an amount of aqueous ascorbic acid solution (0.56 g in 5 g de-ionised water) and aqueous hydrogen peroxide (1.6 g, 35% active) were added to improve the kinetics, and the polymerisation was allowed to continue for a further 30 minutes. The sample was then allowed to cool to room temperature under stirring. The white latex that was obtained consisted of ˜40% solids. The residual vinyl acetate was in the region of 1000 p.p.m., which equates to a conversion of >99.5% of the monomer.

### Example 2

### Effect of Solids Level on Deposition

The above procedure for coating the capsules (10 μm) and measuring the deposition was employed. The capsule dispersions 20% m/m. and 40% m/m. were coated and their deposition compared with the unmodified capsule. The Table below illustrates the results.

| | | Solids | | Rinse 1 | Rinse 2 |
|---|---|---|---|---|---|
| | Coating | % m/m.^{a} | MW Dep % | Ret % | Ret % |
| control | None | N/A. | 35 ± 10 | 30 ± 10 | 30 ± 10 |
| comparative | PVAc- | 20 | 82 ± 5 | 60 ± 5 | 58 ± 5 |
| | LBG | | | | |
| invention | PVAc- | 40 | 92 ± 10 | 78 ± 10 | 76 ± 10 |
| | LBG | | | | |

In the table: MW Dep is deposition obtained after the main wash; R1 Ret is retention on the fabric after the first rinse (i.e. MW dep−R Ret=loss of capsules); R2 Ret is Retention on the fabric after the second rinse. Solids % is starting solids in the dispersion.

It can be seen that the coating of the pre-formed capsule at the higher level of solids led to improved particle deposition when the particles were subsequently used in washing experiments.

### Example 3

### Effect of Reaction Time on Optimization of Particle Deposition

The Table below shows that a selected reaction time for the addition of the coat at 70° C. enhances the deposition of the perfume particles (10 μm).

| | Polymerization | | | |
|---|---|---|---|---|
| Coating | Time/minutes. | MW Dep % | R1 Ret % | R2 Ret % |
| None | N/A | 30 ± 5 | 30 ± 5 | 30 ± 5 |
| (control) | | | | |
| PVAc-LBG | 15 | 25 ± 5 | 20 ± 5 | 17 ± 5 |
| PVAc-LBG | 30 | 60 ± 3 | 50 ± 3 | 42 ± 4 |
| PVAc-LBG | 60 | 60 ± 4 | 50 ± 4 | 46 ± 4 |
| PVAc-LBG | 120 | 63 ± 3 | 55 ± 4 | 50 ± 4 |
| PVAc-LBG | 180 | 57 ± 3 | 43 ± 2 | 40 ± 2 |
| PVAc-LBG | 300 | 37 ± 8 | 30 ± 5 | 28 ± 5 |

In the table: MW Dep is deposition obtained after the main wash; R1 Ret is retention on the fabric after the first rinse (i.e. MW dep−R Ret=loss of capsules); R2 Ret is Retention on the fabric after the second rinse.

It can be seen that best deposition was obtained with a reaction time of ˜120 minutes. It is believed that for the shorter reaction times less LBG is incorporated in the particles and for the longer reaction times the LBG is either digested under the polymerisation conditions or otherwise rendered less effective.

### Example 4

### Improved Deposition as a Function of Monomer Type

Perfume particles (30 μm) were coated as above employing either vinylacetate, methyl acrylate or ethyl acrylate monomers. The reaction parameters were as discussed above. The table below illustrates the results, showing that methyl acrylate afforded capsules with a better deposition profile.

| Coating | MW Dep % | R1 Ret % | R2 Ret % |
|---|---|---|---|
| None (control) | 30 ± 10 | 15 ± 2 | 12 ± 3 |
| PVAC-LBG | 70 ± 5 | 40 ± 2 | 28 ± 3 |
| PMA-LBG | 83 ± 3 | 65 ± 3 | 62 ± 3 |
| PEA-LBG | 55 ± 5 | 38 ± 2 | 32 ± 3 |
| PVAc-XG | 83 ± 20 | 34 ± 10 | 25 ± 7 |

In the table: MW Dep is deposition obtained after the main wash; R1 Ret is retention on the fabric after the first rinse (i.e. MW dep−R Ret=loss of capsules); R2 Ret is Retention on the fabric after the second rinse. PVAC=poly(vinylacetate); PMA=poly(methylacrylate); PEA=poly(ethylacrylate). PVAc=xyloglucan

### Example 5

### Improvement of Capsule Deposition as a Function of PVAc to Capsule

The 10 μm capsules were derivatized with an PVAc-LBG coating via the above method. The monomer (vinyl-acetate) to capsule ratio was altered and the final derivatized capsules deposition measured. The results in the Table below show that the optimum coating ratio was 90:10 capsule:monomer ratio as a function of weight.

| | Ratio | |
|---|---|---|
| | Capsule:Monomer | Main-wash |
| Coat | m:m. | Deposition % |
| None | N/A | 38 ± 2 |
| (control) | | |
| PVAc-LBG | 90:10 | 72 ± 3 |
| PVAc-LBG | 95:5 | 55 ± 4 |
| PVAc-LBG | 99:1 | 37 ± 3 |
| LBG alone | N/A | 28 ± 5 |
| (control) | | |

In the table the Ratio is the weight of perfume capsule to monomer as a function of weight; Deposition is that obtained after main wash; LBG alone is LBG incubated with capsules with no monomer present.

### Example 6

### Improved Stability of Coated Capsules in Detergent and Fabric Conditioner Base

The 10 μm capsules were coated as above (optimized PVAC-LBG) and added to a “Wisk” liquids base (ex Lever Bros), and “Comfort” concentrated fabric conditioner base (ex Lever Bros). After 2 weeks storage at 45° C. several perfumes which have a propensity to leak from capsules when exposed to liquid products were analyzed using GCMS. The table below shows that the PVAc-LBG acts as a barrier to migration of such perfume notes.

| | | % Leakage | | % Leakage |
|---|---|---|---|---|
| | % Leakage | Fabric | % Leakage | “Wisk” |
| | Fabric | Conditioner | “Wisk” | Liquid |
| | Conditioner | PVAc-LBG | Liqyud | PVAc-LBG |
| Perfume Note | No Coat. | coat. | No coat. | coat. |
| Ethyl-2-methyl | 100 | 60 | 77 | 75 |
| pentanoate | | | | |
| Allyl | 85 | 55 | 80 | 60 |
| heptanoate | | | | |
| a-ionone | 90 | 70 | 80 | 60 |
| Undecalactone | 100 | 70 | 80 | 75 |

### Example 7

### Forming Encapsulates with LBG Deposition Aid in a “One-Pot” Reaction

The following perfume components were used to make a simple model perfume:

| | Aldrich | | | |
|---|---|---|---|---|
| | Catalogue | | | b.p. |
| Component | Code | Formula | F.W. | (° C.) |
| α,α-Dimethyl phenethyl | W239208 | C₁₂H₁₆O₂ | 192 | 250 |
| acetate (DMPEA) | | | | |
| Methyl Dihydro Jasmonate | W340804 | C₁₃H₂₂O₃ | 226 | 110 at |
| (MDHJ) | | | | 0.2 mmHg |
| Phenethyl Phenylacetate | W286605 | C₁₆H₁₆O₂ | 325 | 325 |
| (PEPA) | | | | |

The composition of the model perfume was:

| Component | Mass (g) | wt % |
|---|---|---|
| α,α-Dimethyl phenethyl acetate (DMPEA) | 7.8644 | 33.18 |
| Methyl Dihydro Jasmonate (MDHJ) | 7.9347 | 33.26 |
| Phenethyl Phenylacetate (PEPA) | 7.8445 | 33.55 |

Whereas the previous examples had used a simplified system in which the encapsulates were pre-formed, in this example the encapsulates were both formed and modified in situ.

To a 100 ml conical flask was add 19.5 g formalin (37 wt % aqueous formaldehyde) and 44 g water. The pH of the solution was adjusted to 8.9 using 5 wt % Na₂CO₃. 10 g melamine and 0.64 g NaCl were added and stirred for 10 minutes at room temperature. The mixture was heated to 62° C. and stirred until the mixture has become clear. This mixture is hereinafter referred to as “pre-polymer (1)”.

To a 1000 ml polymerisation kettle was added 672 g of water and heated to 75° C., the pre-polymer (1) was added and the pH adjusted to 4.1 using 10 wt % formic acid, after approx 1 minute the solution became turbid.

An Ultraturrax™ (at 8800 rpm) was used to agitate the mixture while slowly adding 112 g of the model perfume over a period of 30 seconds, while continuing to agitate the mixture for a total of 2.5 minutes. The reaction vessel was sealed, heated and stirred at 75° C. for 24 hours. After 24 hours a 20 ml aliquot was removed and the pH adjusted to 7 using 5 wt % Na₂CO₃

A further 10 g of the freshly prepared pre-polymer (1) solution was added to the remaining emulsion, and the pH adjusted to 4.1 using 10 wt % formic acid. 100 ml of a 1% LBG solution in water was then added. The mixture was then left to stir, at 75° C., after 1, 3, 24 hours 20 ml aliquots were removed and adjusted to pH7 using 5 wt % Na₂CO₃.

The resulting derivatized samples were then characterized via their ability to deposit from a main wash solution against the unmodified capsule. The Table below illustrates the results.

| | Extended Reaction | Main wash |
|---|---|---|
| Sample | Time (hours) | Deposition % |
| Unmodified | 0 | 42 |
| (Comparative) | | |
| LBG-MF | 1 | 76 |
| LBG-MF | 3 | 77 |
| LBG-MF | 24 | 76 |

### Example 8

### Control of Viscosity Via Choice of Polysaccharide

The Table below illustrates the impact of employing LBG or Xyloglycan (XG) as the polysaccharide source. The results show that XG produces a lower viscosity system. The processing of the capsules was as stated above, however, the quantities of polysaccharides were as defined in the Table.

| | | | Locust | | Final |
|---|---|---|---|---|---|
| Vinyl | Methyl | Ethyl | Bean | | Viscosity |
| Acetate | Acrylate | Acrylate | Gum/g | Xyloglucan/g | c · Ps. |
| 20 | 0 | 0 | 2 | 0 | 105 |
| 20 | 0 | 0 | 3 | 0 | 206 |
| 20 | 0 | 0 | 4 | 0 | 444 |
| 20 | 0 | 0 | 0 | 2 | 60 |
| 20 | 0 | 0 | 0 | 4 | 190 |
| 20 | 0 | 0 | 0 | 6 | 267 |
| 0 | 20 | 0 | 2 | 0 | 7160 |
| 0 | 0 | 20 | 2 | 0 | 890 |

The LBG or XG were dissolved in 132 g of hot water prior to use. Vinylacetate or methyl acrylate or ethyl acrylate (20 g), perfume encapsulates (30 μm 338 g as 53% m/m. solids).

### Example 9

### Effect of Particle Size on Deposition

5, 15, and 30 μm perfume capsules were coated, and their deposition measured as per above. The Table below illustrates the findings, showing that the smaller particle size had better rinse retention compared to the larger encapsulates.

| Particle | | Main wash | Rinse 1 | Rinse 2 |
|---|---|---|---|---|
| Size | Coating | Deposition % | Retention % | Retention % |
| 5 (control) | None | 22 ± 5 | 20 ± 5 | 20 ± 5 |
| 15 (control) | None | 35 ± 5 | 30 ± 5 | 30 ± 5 |
| 30 (control) | None | 32 ± 10 | 18 ± 5 | 16 ± 5 |
| 5 | PVAc-LBG | 95 ± 2 | 90 ± 2 | 85 ± 5 |
| 15 | PVAc-LBG | 95 ± 5 | 71 ± 3 | 70 ± 2 |
| 30 | PVAc-LBG | 80 ± 3 | 54 ± 4 | 32 ± 5 |

Main wash deposition is the deposition obtained after the main wash. Rinse retention 1 is the retention on the fabric after the first rinse (i.e. Main wash deposition-Rinse retention 1=loss of capsules); Rinse retention 2 is the retention on the fabric after the second rinse (i.e. Main wash deposition-Rinse retention=loss of capsules).

### Example 10

### Enhancing Top Note Delivery

The following table illustrates the enhanced delivery of top note from the PVAc-LBG coated particle after a machine wash (Miele 40° C. cotton cycle) and line drying for two days:

| | Top Note - | | Base Note - |
|---|---|---|---|
| | Ethyl 2-methyl | Mid Note - | Isopropyl |
| | pentanoate | α-Ionone | Myristate |
| | (GC-MS | (GC-MS | (GC-MS |
| Coating | Peak Area) | Peak Area) | Peak Area) |
| Free | 0 | 0 | 180282 |
| Perfume | | | |
| Capsule | 0 | 498584 | 1202182 |
| without | | | |
| PVAc-LBG | | | |
| Capsule | 190357 | 849671 | 1691836 |
| with | | | |
| PVAc-LBG | | | |

N.B. Free perfume and non coated capsule were dosed at 1% and PVAc-LBG at 0.5% (on powder formulation).

1. A process for the manufacture of core-shell particles by emulsion polymerisation wherein the core comprises a perfume and the shell comprises a non-ionic deposition aid which is substantive to textiles characterised in that during the emulsion polymerisation step the solids content does not fall below 25% wt.

2. A process according to claim 1 wherein the solids content is 30-50% wt throughout the polymerisation process.

3. A process according to claim 1 which comprises a first process step comprising the formation of perfume encapsulates comprising a perfume core and shell from an emulsion of perfume in an aqueous solution of a monomer.

4. A process according to claim 3 in which said first process step is a step-growth polymerisation.

5. A process according to claim 4 wherein the monomer includes melamine/urea (or mixtures thereof) and formaldehyde monomers.

6. A process according to claim 3 in which said first process step is an addition polymerisation.

7. A process according to claim 6 wherein the monomer includes methyl methacrylate.

8. A process according to claim 1 which comprises a second process step comprising the attachment of the non-ionic deposition aid to a particle.

9. An aqueous dispersion of particles obtainable by a process according to claim 1, wherein said dispersion has a solids content of above 25%.

10. Use of a dispersion according to claim 9 in a process for the manufacture of a laundry detergent composition.
